(19) United States
(12) Patent Application Publication
(10) Pub. No.: US 2020/0349822 A1
(43) Pub. Date: Nov. 5, 2020

(54) FALL DETECTION USING THE TRIBOELECTRIC EFFECT

(71) Applicant: KONINKLIJKE PHILIPS N.V., EINDHOVEN (NL)

(72) Inventors: Mark Thomas JOHNSON, Arendonk (BE); Neil Francis JOYE, Waalre (NL); Warner Rudolph Theophile TEN KATE, Waalre (NL)

(21) Appl. No.: 16/758,542

(22) PCT Filed: Oct. 24, 2018
(86) PCT No.: PCT/EP2018/079074

§ 371 Date: Apr. 23, 2020

Related U.S. Application Data
(60) Provisional application No. 62/578,032, filed on Oct. 27, 2017.

(51) Int. Cl. G08B 21/04; A61B 5/11

(52) U.S. Cl. CPC G08B 21/0446; A61B 2560/04; A61B 5/1117; G08B 21/043

(57) ABSTRACT

The present disclosure is directed to methods and apparatus for leveraging the triboelectric effect to improve fall detection. In various embodiments, fall detection system (100) may include: a triboelectric sensor (102) that is securable to a portion of a person's body or is affixed to clothing worn by the person, wherein the triboelectric sensor includes at least one electrode (103) that defines a surface; readout circuitry (104) that detects charge at the at least one electrode caused by physical contact between the surface of the at least one electrode and at least one other surface; and logic (106) that receives, from the readout circuitry, a first signal indicative of the detected charge at the at least one electrode and, based at least in part on the first signal, provides output indicative of a detected fall.

### TECHNICAL FIELD

Various embodiments described herein are directed generally to health care. More particularly, but not exclusively, various methods and apparatus disclosed herein relate to leveraging the triboelectric effect to improve fall detection.

### BACKGROUND

The ability to detect when a person has fallen is important in a variety of contexts, such as disaster relief, firefighting, and in particular, elderly care. Falls by elderly patients may cause severe injuries such as hip fractures, especially in patients with osteoporosis. These injuries can in some cases be immediately fatal, and even when not immediately fatal may trigger a gradual deterioration of the patient. In general, severe adverse effects can be reduced by providing assistance quickly. Mechanisms exist for detecting falls in the elderly care context. For example, fall sensors worn as pendant devices around patients' necks or attached to patients' torsos tend to provide fairly reliable fall data. However, deploying fall sensors at these locations may be intrusive and/or uncomfortable for the patients, and deploying them elsewhere on patients may lead to less reliable fall data. For example, it is harder to design a fall detector worn at the wrist (e.g., using sensors built-in a watch) that exhibits accuracy comparable to necklace-based fall detectors. Moreover, many fall detection sensors measure a change in patient orientation to detect a fall. However, elderly patients' orientations often do not change when they fall. For example, when elderly patients fall at their bedsides, they often end up on the floor in a sitting posture, and such an upright posture may not be interpreted as a fall, especially if other signals raised by other sensors (e.g., accelerometers) do not corroborate a fall. The fall detection accuracy can be improved when it is known the user is sitting or lying, since parameters like the height drop and orientation change, as measured by an (accelerometer) sensor at the torso, for example, can be conditioned on the determined ending posture.

### SUMMARY

The present disclosure is directed to methods and apparatus for leveraging the triboelectric effect to improve fall detection. In various embodiments, a triboelectric sensor may be deployed at various locations relative to a person, e.g., on the person's clothing or affixed to the person's body. When one or more electrodes of the triboelectric sensor make contact with surface (e.g., a floor, the ground) with different triboelectric characteristics, electrons may be exchanged between one or more of electrodes and the surface. Depending on the particular configuration of the electrode (several variations are described herein), the electrode of the triboelectric sensor may end up building up triboelectric charges (e.g., voltage, current) that can be detected, e.g., by readout circuitry (which may include, for instance, one or more amplifiers). The readout circuitry may raise a signal indicative of the voltage/current (or triboelectric charge) built up on the electrode and/or indicative of a charge leakage from the electrode. Logic may provide output indicative of a fall based on such a signal.

Generally, in one aspect, A fall detection system may include: a triboelectric sensor that is securable to a portion of a person's body or is affixed to clothing worn by the person, wherein the triboelectric sensor includes at least one electrode that defines a surface; readout circuitry that detects charge at the at least one electrode caused by physical contact between the surface of the at least one electrode and at least one other surface; and logic that receives, from the readout circuitry, a first signal indicative of the detected charge at the at least one electrode and, based at least in part on the first signal, provides output indicative of a detected fall.

In various embodiments, the passive triboelectric sensor may be affixed to an inner surface of the clothing, and the at least one other surface comprises skin of the person. In various versions, the passive triboelectric sensor may be interwoven into the clothing. In various embodiments, the passive triboelectric sensor may be affixed to an outer surface of the clothing. In various embodiments, the passive triboelectric sensor may be incorporated with a hip protector adorned by the person.

In various embodiments, the at least one electrode may include a dielectric outer surface. In various embodiments, the at least one electrode may include includes two or more electrodes. In various versions, a first electrode of the two or more electrodes may include a dielectric outer surface, a second electrode of the two or more electrodes may not include a dielectric outer surface and is connected to ground, and the first and second electrodes may be arranged to contact the at least one other surface approximately simultaneously. In various versions, a first electrode of the two or more electrodes may tend to become positively charged, and a second electrode of the two or more electrodes may tend to become negatively charged.

In various embodiments, the fall detection system may further include one or more ground connections (413), wherein additional downward pressure of the triboelectric sensor after initial contact causes the one or more ground connections to come into contact with the at least one other surface, thereby discharging the at least one other surface.

In various embodiments, the fall detection system may further include means for physically separating the surface of the at least one electrode from the at least one other surface immediately after the physical contact between the surface of the at least one electrode and at least one other surface. In various embodiments, the fall detection system may further include an accelerometer that provides a second signal indicative of movement by the person—the logic may provide the output indicative of a fall further based on the second signal. In various embodiments, the fall detection system may further include an air pressure sensor that provides a second signal indicative of a detected change in air pressure caused by movement by the person—the logic may provide the output indicative of a fall further based on the second signal. In various embodiments, the readout circuitry includes a peak detector.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

### BRIEF DESCRIPTION OF THE DRAWINGS

In the drawings, like reference characters generally refer to the same parts throughout the different views. Also, the drawings are not necessarily to scale, emphasis instead generally being placed upon illustrating various principles of the embodiments described herein.

FIG. 1A schematically depicts example components that may be employed to practice techniques described herein, in accordance with various embodiments.

FIGS. 1B and 1C depict various locations on which a triboelectric sensor and other components described herein may be disposed relative to people, in accordance with various embodiments.

FIGS. 2A, 2B and 2C depict one example of how techniques described herein may be implemented, in accordance with various embodiments.

FIGS. 3A, 3B, 3C, 3D, and 3E depict multiple examples of how techniques described herein may be implemented, in accordance with various embodiments.

FIGS. 4A, 4B, and 4C depict another example of how techniques described herein may be implemented, in accordance with various embodiments.

FIGS. 5A and 5B depict one example of how techniques described herein may be implemented, in accordance with various embodiments.

FIGS. 6A and 6B depict two examples of how readout circuitry may be implemented, in accordance with various embodiments.

FIG. 7 depicts an example method for practicing selected aspects of the present disclosure.

### DETAILED DESCRIPTION

The ability to detect when a person has fallen is important in a variety of contexts, such as disaster relief, firefighting, and in particular, elderly care. Falls by elderly patients may cause severe injuries such as hip fractures, especially in patients with osteoporosis. These injuries can in some cases be immediately fatal, and even when not immediately fatal may trigger a gradual deterioration of the patient. Mechanisms exist for detecting falls in the elderly care context. For example, fall sensors worn as pendant devices around patients' necks or attached to patients' torsos tend to provide fairly reliable fall data. However, deploying fall sensors at these locations may be intrusive and/or uncomfortable for the patients, and deploying them elsewhere on patients may lead to less reliable fall data. Moreover, many fall detection sensors measure a change in patient orientation to detect a fall. However, elderly patients' orientations often do not change when they fall. For example, when elderly patients fall at their bedsides, they often end up on the floor in a sitting posture, and such an upright posture may not be interpreted as a fall, especially if other signals raised by other sensors (e.g., accelerometers) do not corroborate a fall.

In view of the foregoing, various embodiments and implementations of the present disclosure are directed to leveraging the triboelectric effect to improve fall detection. While examples set forth herein relate to elderly care, this is not meant to be limiting. In various embodiments, techniques, devices, and systems described herein may be applicable in other contexts, such as disaster relief, firefighting, law enforcement, the military, and any other context in which it may be desirable to detect when a person has fallen.

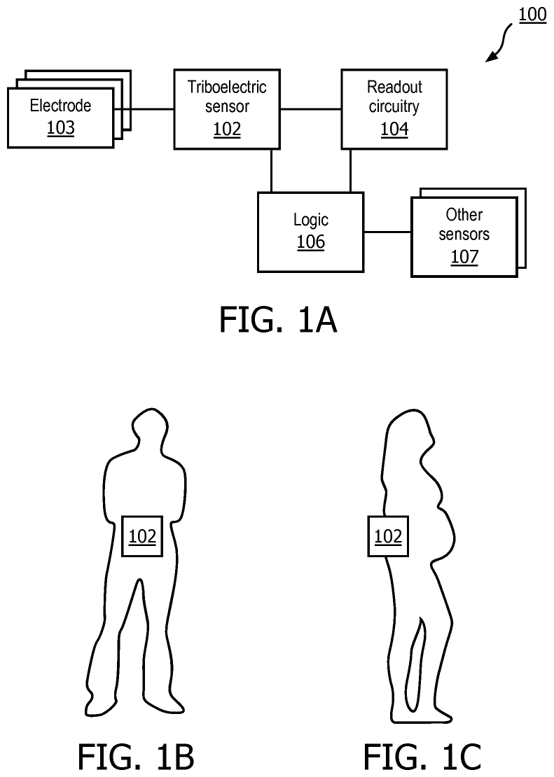

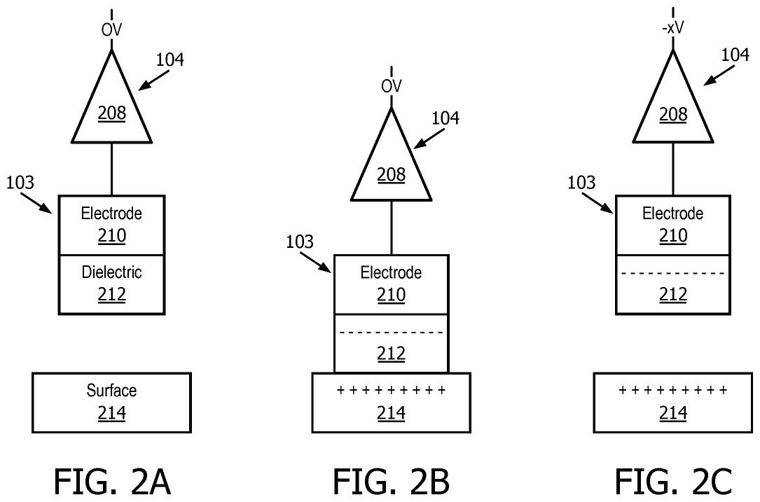

FIG. 1A schematically depicts example components that may be employed to practice techniques described herein, in accordance with various embodiments. A fall detection system 100 may include a triboelectric sensor 102, readout circuitry 104, and logic 106, each which may be operably coupled to one or more of the others using various types of communication technology, such as via one or more busses, wireless communications (e.g., personal area networks such as Bluetooth, ZigBee, Wi-Fi, etc.), various wired technologies, and so forth. In some embodiments, components of fall detection system 100 may be integral within a single housing (not depicted). In other embodiments, one or more of the components of fall detection system 100 may be distributed amongst multiple form factors.

Triboelectric sensor 102 may take various forms. In some embodiments, triboelectric sensor 102 may include one or more electrodes 103. Triboelectric sensor 102 may be configured to provide an electrical signal such as voltage, current and/or charge generated at one or more of the electrodes 103 by way of the triboelectric effect. The triboelectric effect (also known as triboelectric charging) is a contact-induced electrification in which a material becomes electrically charged after it is contacted with a different material through friction. Triboelectric generation is based on converting mechanical energy into electrical energy through methods which couple the triboelectric effect with electrostatic induction. Triboelectric charging is also referred to as static electricity. As a non-limiting example, amber acquires an electrical charge when contacted with (e.g., rubbed against) with materials such as wool. The triboelectric effect also may be observed in a repetitive fashion when a person rubs a balloon against his or her hair. When the balloon is withdrawn, the balloon and the person's hair have opposite charges, which causes the hair and balloon to be attracted to each other. More particularly, electrons from the person's hair are transferred to the balloon during contact, which results in the balloon having an excess of electrons and therefore being charged negatively. Likewise, the person's hair has a shortage of electrons and therefore is positively charged.

As will be discussed in more detail shortly, each electrode 103 of triboelectric sensor 102 may be constructed with various materials having various triboelectric properties, i.e. according to the tendency of the materials to gain electrons (become negatively charged) or lose electrons (become positively charged). In some embodiments, one or more of the electrodes 103 may include a dielectric outer surface. In various embodiments, one or more of the electrodes 103 may include a substantially flat and/or conformable surface. This flat and/or conformable surface may contact a person's skin or the floor (or the ground) when the person falls. Triboelectric sensor 102 may be deployed at various locations relative to a person. For example, in FIG. 1B, triboelectric sensor 102 is deployed proximate a person's hips, e.g., as an integral part of a hip protector adorned by the person or as part of clothing worn by the person. As another example, in FIG. 1C, triboelectric sensor 102 is deployed proximate the person's buttocks, as it is common for people to fall on their buttocks.

Referring back to FIG. 1A, readout circuitry 104 may be configured to detect voltage or current at one or more of the electrodes 103 caused by physical contact between the flat surface of the at least one electrode and at least one other surface (e.g., the wearer's skin, the floor, the ground, etc.), and to provide a signal indicative of the detected voltage or current to logic 106. Logic 106 may be configured to receive, e.g., from readout circuitry 104, a signal indicative of the detected voltage or current at the at least one electrode 103. Based at least in part on the signal provided by readout circuitry 104, logic 106 may provide output indicative of a detected fall. For example, logic 106 may cause a display or audio component of a computing device carried by the wearer or remote therefrom to provide a visual and/or audio indication of a detected fall. In some embodiments, the output may be provided at a computing device deployed at a nursing station and/or other base station in communication with logic 106, so that medical personnel, caregivers, and/or the like may be informed of falls immediately and can respond appropriately.

While depicted separately in FIG. 1A, readout circuitry 104 and logic 106 may in some embodiments form a single unit. Logic 106 (and/or readout circuitry 104) may take various forms, such as an application-specific integrated circuit (“ASIC”) or a field-programmable gate array (“FPGA”). In other embodiments, logic 106 may include one or more processors, such as one or more central processing units (“CPU”), one or more graphics processing units (“GPU”), one or more microprocessors, etc., that are configured to execute instructions stored in memory (not depicted). For example, in some embodiments, logic 106 may take the form of one or more processors contained in a person's mobile phone, wearable device (e.g., smart watch and smart glasses), and so forth. In some such embodiments, a signal produced by triboelectric sensor 102 may be transmitted to logic 106 using various types of wireless communication, such as low-energy Bluetooth (BLE), ZigBee, etc.

In various embodiments, fall detection system 100 may include other components, such as other sensor(s) 107 operably coupled and/or in communication with logic 106, which may aide in the detection of falls. For example, in some embodiments, fall detection system 100 may include an accelerometer that may be variously configured to monitor for abrupt changes in the wearer's orientation that may signify a fall, estimate a height drop (e.g., by double integrating the acceleration signal), and/or monitor a magnitude of an impact, and to provide a signal indicative thereof, e.g., to logic 106. In some such cases, the accelerometer may be a triaxial accelerometer configured to provide a signal indicative of acceleration in three different linear directions, including the direction of gravity. Additionally or alternatively, in some embodiments, fall detection system 100 may include a gyroscope that provides angular velocity of the wearer. Additionally or alternatively, in some embodiments, fall detection system 100 may include a barometric (e.g., air) pressure sensor that, for instance, may provide a signal that constitutes surrogate measure of altitude. Additionally or alternatively, in yet other embodiments, fall detection system 100 may include other types of sensors, such as photoplethysmogram (“PPG”) sensors to detect parameters such as skin conductivity (and which may be co-located with triboelectric sensor 102). Signals from one or more of these other sensors may be used, e.g., in conjunction with signals from triboelectric sensors, to detect falls. For example, in some embodiments, a signal from one sensor (e.g., triboelectric sensor 102) may be used to corroborate a fall signal produced by one or more other sensors 107 (e.g., a triaxial accelerometer and a barometric pressure sensor), or vice versa. It should be understood that any combination of signals from any combination of the aforementioned sensors may be used collectively and/or corroboratively to detect falls.

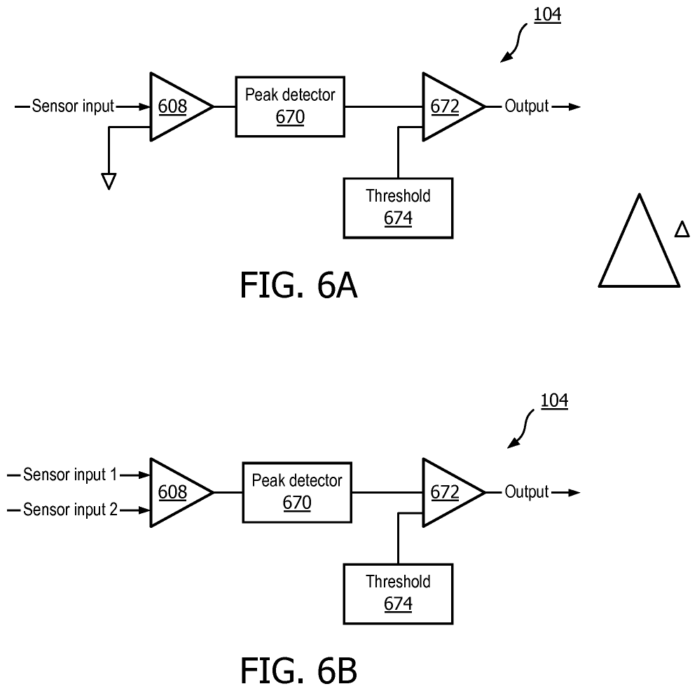

In some embodiments, readout circuitry 104 may include one or more amplifiers (not depicted in FIGS. 1A-C) that may or may not have relatively high impedance (e.g., Z=200 TΩ). In some embodiments, triboelectric sensor 102 may be passive, and this fact coupled with high-impedance readout circuitry 104 may enable low current consumption, which in turn may lead to reduced power consumption. This may constitute a technical advantage in that a person that uses fall detection system 100 may not need to frequently recharge the system 100. In embodiments in which current is measured to detect a fall, instead of voltage, the amplifier may have a relatively low impedance. FIGS. 6A-B depict two non-limiting examples of how readout circuitry 104 may be implemented.

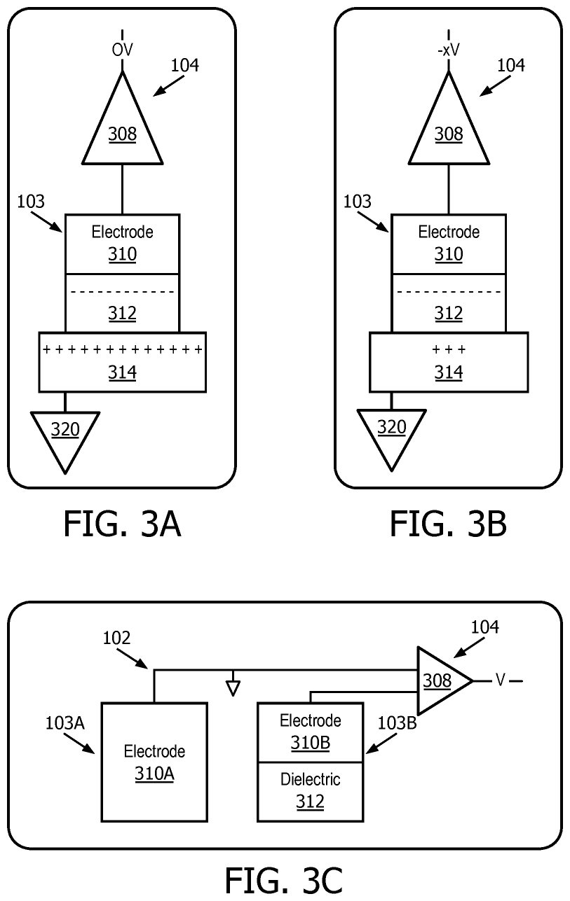

Referring now to FIGS. 2A-C, an example is demonstrated of what happens when an “ideal” (e.g., completely electrically insulated) electrode 103 of triboelectric sensor 102 (see FIG. 1A) is brought into contact with a surface 214 such as a floor (e.g., with carpet) or the ground, and then removed from surface 214. In some cases, surface 214 may be considered a positive material (i.e. becomes positively charged), such as wool carpet, whereas a dielectric outer surface 212 may be considered a negative material (i.e. becomes negatively charged), such as Teflon. Amplifier 208 may have a relatively large impedance, e.g., due to it being an open circuit. Although not depicted in the Figures, in some embodiments, amplifier 208 may include a power supply such a battery, which in some cases may be rechargeable.

In this example, readout circuitry 104 includes an amplifier 208 that detects voltage/current generated at electrode 103, e.g., with respect to a reference voltage or current (e.g., ground). Electrode 103 in this example includes what will be referred to herein as a “sub” electrode 210 (which in many cases may be electrically conductive) with a dielectric outer surface 212. In FIG. 2A, electrode 103 has not yet contacted surface 214. Consequently, electrode 103 remains uncharged and zero volts are read from amplifier 208.

As illustrated in FIG. 2B, electrode 103, and more particularly, dielectric outer surface 212, may be in contact with surface 214. Consequently, and due to different nominal charges of surface 214 and dielectric outer surface 212, electrons may be exchanged between dielectric outer surface 212 and surface 214 due to the triboelectric effect. As a result, surface 214 is now positively charged and dielectric outer surface 212 is negatively charged. However, while dielectric outer surface 212 and surface 214 remain in contact, in many scenarios, the charges between surface 214 and dielectric outer surface 212 may remain balanced. Thus, the voltage readout from amplifier 208 remains at zero.

In FIG. 2C, electrode 103 has been removed from surface 214. Consequently, the negative charges (i.e. excess electrons) at dielectric outer surface 212 are no longer balanced by the positive charges (i.e., electron shortage) of surface 214. This effect is known as the electrostatic induction. Thus, the voltage readout from amplifier 208 is now a negative voltage.

What can be seen from FIGS. 2A-C is that a typical triboelectric sensor does not generate voltage when it comes into contact with surface 214, but rather when it is released from surface 214. This is due to the fact that while in contact, there is a charge balance between dielectric outer surface 212 and surface 214. Thus, in some embodiments, an arrangement such as that depicted in FIGS. 2A-C may be used, for instance, to determine whether a wearer has gotten up after a potential fall. This might be used to revoke sending a fall alert to a care giver. The user is able to move therefore could call for help using an explicit alarm such as a help (push) button. In some embodiments, this fact—that the wearer has risen very shortly after falling—may be used to corroborate the fact of the wearer's fall because if the wearer had meant to stay down, they likely would not have risen so suddenly.

However, with other embodiments described herein, it may be desirable to measure voltage and/or current at contact between electrode 103 and surface 214, in addition to or instead of after release. This may be accomplished in various ways. For example, in some scenarios, surface 214 may dissipate its collected charges relatively quickly, e.g., as though it were connected to ground (in some scenarios surface 214 very well may be the actual ground). Additionally or alternatively, in some embodiments, dielectric outer surface 212 may be omitted, so that electrode 103 only includes the “sub” electrode 210—this may perform particularly well in scenarios where surface 214 is constructed with a strongly positive or negative material (assuming sub electrode 210 is the opposite charge). Additionally or alternatively, in some embodiments, various mechanisms may be employed to ensure that dielectric outer surface 212 (or electrode 103 as a whole) breaks contact with surface 214 shortly after impact, thereby generating measureable voltage/current that can be used to detect a fall.

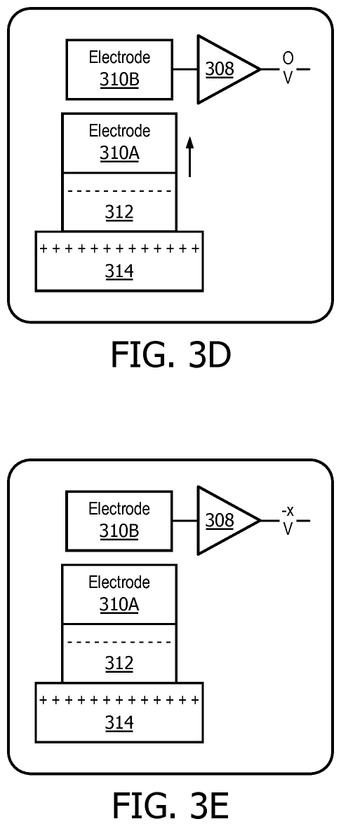

Referring now to FIGS. 3A and 3B, an alternative arrangement to that of FIGS. 2A-C is depicted in which readout circuitry 104 once again includes an amplifier 308, and electrode 103 once again includes a sub electrode 310 and a dielectric outer surface 312. In this example, surface 314 (again, may be carpet, tile, other types of flooring, etc.) is not able to retain its surface charges due to charge leakage, as indicated by the schematic ground 320. This in fact is fairly realistic, as the resistance to ground for most floor surfaces is very low. In some embodiments, assuming the charges acquired on surface 314 during contact (3A) dissipate quickly enough, then dielectric surface 312 may acquire a net negative charge (3B) even before contact between dielectric surface 312 and surface 314 is broken. Thus, as is shown in FIG. 3B, a negative voltage can be measured even before the physical interface between dielectric outer surface 312 and surface 314 is broken, e.g., very shortly after contact is initially made.

As another example, FIG. 3C depicts an embodiment in which readout circuitry 104 once again includes an amplifier 308, but where the one or more electrodes of triboelectric sensor 102 includes a first sub electrode 310A and a second sub electrode 310B, e.g., arranged to make simultaneous contact with the underlying surface (not depicted in FIG. 3C). In this example, second sub electrode 310B includes a dielectric outer surface 312, whereas first sub electrode 310A does not. In some embodiments, sub electrodes such as first sub electrode 310A and/or second sub electrode 310B are constructed with metal and/or with materials different from each other. In some embodiments, the metal sub electrode 310A may function to discharge a contact surface (not depicted in FIG. 3C, 314 in other Figs.), such that a charge imbalance develops quickly between dielectric outer surface 312 and the contact surface. As explained previously, this quickly-acquired charge can be detected, e.g., by amplifier 308. In some embodiments, first sub electrode 310A may be a ring electrode around triboelectric sensor 102.

FIGS. 3D and 3E depict an embodiment in which a three-layer system, as opposed to the parallel configuration of FIG. 3C, is employed. Contact with the surface 314 (e.g., floor, carpet, etc.) first causes charging of a dielectric outer surface 312, but then urges a first sub electrode 310A towards a second sub electrode 310B (as indicated by the upward arrow in FIG. 3D). Once contact is made between the first sub electrode 310A and the second sub electrode 310B (or a ground connection), charges collected by the dielectric outer surface 312 may be discharged through the amplifier 308 to produce an electrical signal. This signal is measurable since the dielectric-metal diffusion between the dielectric outer surface 312 and one or both sub electrodes 310A/310B is a relatively slow process.

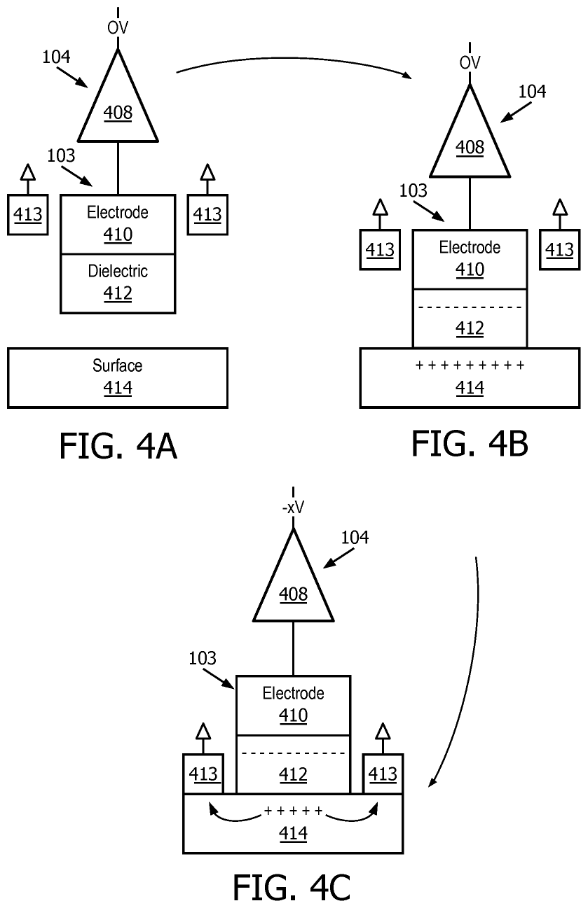

FIGS. 4A-C depict an embodiment similar to that depicted in FIGS. 3D-E in some respects. In FIG. 4A, electrode 103, which includes both sub electrode 410 and dielectric outer surface 412, has not yet contacted surface 414 (e.g., floor, carpet, etc.). Electrode 103 includes one or more ground connections 413 that may be mechanically and/or electrically coupled with electrode 103. In FIG. 4B, dielectric outer surface 412 has contacted surface 414, such that dielectric outer surface 412 is now negatively charged (e.g., has excess electrons) and surface 414 is positively charged. However, because the charges are balanced, no voltage is yet measured at the output of amplifier 408. In FIG. 4C, additional downward pressure has caused the one or more ground connections 413 to come into contact with surface 414, thereby discharging the positive charge of surface 414. Consequently, in FIG. 4C, a negative voltage is measured at the output of amplifier 408.

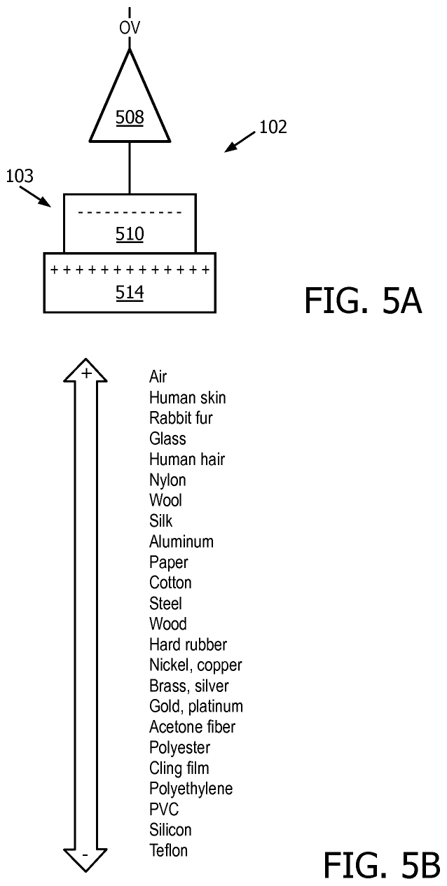

FIG. 5A depicts another variation that operates well for falls that cause contact with very positive or negative surfaces, such as skin or hair. In FIG. 5A, triboelectric sensor 102 only includes a single (e.g., electrically conductive) sub electrode 510—there is no dielectric outer surface. In this example, when electrode 103 contacts surface 514, which may be skin in many scenarios, the triboelectric effect may occur between surface 514 and sub electrode 510. Sub electrode 510 may be directly connected to amplifier 408. Thus, a voltage may be measured upon contact between sub electrode 510 and surface 514.

FIG. 5B depicts a triboelectric series that demonstrates a spectrum from generally positive materials, i.e. materials than tend to become positively charged (with air being the most positive) to generally negative materials, i.e. materials that tend to become negatively charged (with Teflon being the most negative). It can be seen that some metals such as steel and aluminum are situated relatively close to the middle of the spectrum. Thus, if sub electrode 510 of FIG. 5A is constructed with metal, then the embodiment of triboelectric sensor 102 that is depicted in FIG. 5A would likely function most effectively when material 514 is strongly positive or negative, such as when it is human skin and/or hair. Or, if sub electrode 510 of FIG. 5A is deployed on the outside of a wearer's clothing, in some embodiments, multiple electrodes may be deployed in parallel (i.e. so that they contact surface 514 approximately simultaneously), each constructed with a different material and thus able to create charge when contacted with multiple different floor materials. For example, in some embodiments, one sub electrode 510 may be gold or platinum (tending to be/become negatively charged), and another may be, for instance, aluminum (tending to be/become positively charged).

As another variation, in some embodiments, one or more electrodes 103 of triboelectric sensor 102 may be configured to break contact with the operative surface immediately after initial contact. For example, if the operative surface is the floor, one or more electrodes 103 of triboelectric sensor 102 may be mechanically urged, e.g., using one or more springs or force-inducing materials, to break contact with the floor soon after initial contact, even if the wearer is unable to rise. In some such embodiments, one or more springs or other similar mechanisms may be provided to cause an oscillating (or repeating) “bounce,” which can be detected by way of oscillating changes to detected voltage/current and used, for example, as a fall signature. In addition to generating a fall signature, the repeated contact and separation may generate more positive or negative charge, e.g., on a dielectric outer surface, which may be easier to detect. In other embodiments, the clothing to which the triboelectric sensor 102 is secured may be constructed with sponge-like materials that tend to expand after an initial compression, such as neoprene, Styrofoam, rubber (e.g., with inner air pockets), and so forth. In some embodiments, a mechanism similar to bubble packaging often used during shipping may be employed, e.g., between layers of multi-layer clothing worn by a patient.

As alluded to above, in various embodiments, one or more electrodes 103 of triboelectric sensor 102 may take various forms and may be deployed at various positions relative to a person for which fall detection is desired. In some embodiments, one or electrodes 103 may include at least a flat surface that may or may not be conformable, and that is meant to be physically contacted with another surface (e.g., 214, 314, 414), which as noted above could be various forms of floor. However, in other embodiments, one or more electrodes may be intended to be physically contacted with a wearer's skin (which tends to become positively charged).

An added functionality of such a multi-electrode embodiment is that it may be capable of differentiating between the surfaces which are contacted, e.g., by the wearer during the fall. For example, suppose the wearer has nylon carpets and furniture made of other, non-nylon materials. It may be possible to distinguish between situations in which the wearer sits in a non-nylon chair and when the wearer contacts a nylon carpet during a fall. This may be established, for instance, by the detected polarity or amplitude of the signal generated by readout circuitry 104.

In some such embodiments, more than one electrode 103 may be employed (e.g., FIG. 3C), such that the voltage potential difference between the multiple electrodes 103 can be measured. If different dielectric materials are deposited on each of the multiple electrodes, with each deposited material having different triboelectric properties, then the material with which each electrode makes contact during a fall can be determined with heightened precision.

In some embodiments in which one or more electrodes 103 of triboelectric sensor 102 are positioned on an outer surface of a wearer's clothing, the electrodes may be either attached to the outer surface with stitching and/or adhesive, or may be interwoven into the clothing (e.g., as positive or negative threads). In either case, the triboelectric sensor 102 may serve to detect contact between one or more of the electrodes 103 and a surface such as a floor. In some such embodiments, the one or more electrodes may be constructed with materials that have triboelectric properties that differ from typical flooring material. Floors, whether carpeted or not, often include materials such as nylon, wool, wood, and/or stone. As shown in FIG. 5B, these common flooring materials may tend towards the positive end of the triboelectric spectrum.

In some embodiments, one or more electrodes 103 of triboelectric sensor 102 may be incorporated onto a waistband, which may be worn in some cases over the wearer's underwear but beneath their outer clothing. As was the case with other embodiments, the one or more electrodes 103 may be attached to an outer surface of the waistband and/or interwoven into the waistband. Such embodiments may thereby cover the wearer's hip area for fall detection purposes.

FIGS. 6A and 6B depict examples of how readout circuitry 104 may be implemented, in accordance with various embodiments. In FIG. 6A, a sensor input, e.g., from triboelectric sensor 102, may be provided as input to an amplifier 608, along with ground as another input. The output of amplifier 608 may be provided as input to a peak detector 670. Peak detector 670 may in some embodiments include a high pass filter. The output of peak detector 670 is provided, along with some threshold 674, as input to a comparator 672 for comparison. In various embodiments, output of comparator 672, which may or may not be provided to logic 106, may be indicative of whether a particular threshold voltage is satisfied. As noted above, comparator 672 and/or threshold 674 may be part of logic 106.

In FIG. 6B, two sensor signals, e.g., provided by two electrodes 103A and electrode 103B of FIG. 3C, may be provided as input to amplifier 608. The output of amplifier 608 may be provided as input for peak detector 670. The output of peak detector 670 may once again be compared by comparator 672 to a threshold 674. The output of comparator 672 may be similar as in FIG. 6A. In some embodiments, amplifier 608 may be implemented as a differential amplifier, such that a voltage/current difference between the two sensor inputs can be measured.

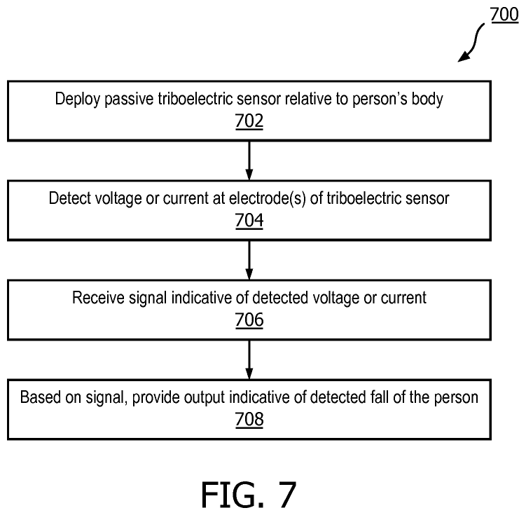

FIG. 7 depicts an example method 700 for practicing selected aspects of the present disclosure, in accordance with various embodiments. While operations of method 700 are shown in a particular order, this is not meant to be limiting. One or more operations may be reordered, omitted or added.

At block 702, a passive triboelectric sensor (e.g., 102) may be deployed relative to a portion of a person's body. As noted above, the passive triboelectric sensor may be deployed at various locations relative to the person, such as near their hips or buttocks. Additionally, the triboelectric sensor may be deployed in various ways, such as being affixed to clothing, woven into clothing, inserted between layers of multi-layer clothing, incorporated into a hip protector, and so forth. As described previously, in various embodiments, the passive triboelectric sensor may include at least one electrode that defines a flat surface.

At block 704, readout circuitry (e.g., 104) that is operably coupled with the passive triboelectric sensor may detect voltage or current at the at least one electrode that is caused by physical contact between the flat surface of the at least one electrode and at least one other surface (e.g., a floor, the ground, etc.). The voltage and/or current may be detectable due to deployment of one or more of the various configurations described above.

At block 706, logic (e.g., 106) operably coupled with the readout circuitry may receive a signal indicative of the detected voltage or current at the at least one electrode. Based at least in part on the signal, at block 708, the logic may provide output that is indicative of a detected fall of the person. In some embodiments, the logic may be operably coupled with an output component such as a display or speaker, and may cause one or more of such output components to provide audio and/or visual output that includes notification of the detected fall. Additionally or alternatively, in some embodiments the logic may be operably coupled with a wired and/or wireless communication interface. The logic may use such a communication interface to transmit data indicative of the detected fall to a remote computing device, such as a computing device operated and/or carried by a caregiver, so that the caregiver may be notified of the detected fall.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of and “consisting essentially of” shall be closed or semi-closed transitional phrases, respectively, as set forth in the United States Patent Office Manual of Patent Examining Procedures, Section 2111.03. It should be understood that certain expressions and reference signs used in the claims pursuant to Rule 6.2(b) of the Patent Cooperation Treaty (”PCT″) do not limit the scope.

1. A fall detection system comprising:
a triboelectric sensor that is securable to a portion of a person's body or is affixed to clothing worn by the person, wherein the triboelectric sensor includes at least one electrode that defines a surface;
readout circuitry that detects charge at the at least one electrode caused by physical contact between the surface of the at least one electrode and at least one other surface; and
logic that receives, from the readout circuitry, a first signal indicative of the detected charge at the at least one electrode and, based at least in part on the first signal, provides output indicative of a detected fall.

2. The fall detection system of claim 1, wherein the triboelectric sensor is affixed to an inner surface of the clothing, and the at least one other surface comprises skin of the person.

3. The fall detection system of claim 1, wherein the triboelectric sensor is interwoven into the clothing.

4. The fall detection system of claim 1, wherein the triboelectric sensor is affixed to an outer surface of the clothing.

5. The fall detection system of claim 1, wherein the triboelectric sensor is incorporated with a hip protector adorned by the person.

6. The fall detection system of claim 1, wherein the at least one electrode includes a dielectric outer surface.

7. The fall detection system of claim 1, wherein the at least one electrode includes two or more electrodes.

8. The fall detection system of claim 7, wherein a first electrode of the two or more electrodes includes a dielectric outer surface, a second electrode of the two or more electrodes does not include a dielectric outer surface and is connected to ground, and the first and second electrodes are arranged to contact the at least one other surface approximately simultaneously.

9. The fall detection system of claim 7, wherein a first electrode of the two or more electrodes tends to become positively charged, and a second electrode of the two or more electrodes tends to become negatively charged.

10. The fall detection system of claim 1, further comprising one or more ground connections, wherein additional downward pressure of the triboelectric sensor after initial contact causes the one or more ground connections to come into contact with the at least one other surface, thereby discharging the at least one other surface.

11. The fall detection system of claim 1, further comprising means for physically separating the surface of the at least one electrode from the at least one other surface immediately after the physical contact between the surface of the at least one electrode and at least one other surface.

12. The fall detection system of claim 1, further comprising an accelerometer that provides a second signal indicative of movement by the person, wherein the logic provides the output indicative of a fall further based on the second signal.

13. The fall detection system of claim 1, further comprising an air pressure sensor that provides a second signal indicative of a detected change in air pressure caused by movement by the person, wherein the logic provides the output indicative of a fall further based on the second signal.

14. The fall detection system of claim 1, wherein the readout circuitry includes a peak detector.

15. A method for detecting when a person falls, comprising:
deploying a triboelectric sensor relative to a portion of the person's body, wherein the triboelectric sensor includes at least one electrode that defines a surface;
detecting, by readout circuitry that is operably coupled with the triboelectric sensor, charge at the at least one electrode caused by physical contact between the surface of the at least one electrode and at least one other surface;
receiving, by logic operably coupled with the readout circuitry, a first signal indicative of the detected charge at the at least one electrode; and
based at least in part on the first signal, providing, by the logic, output that is indicative of a detected fall of the person.

16. The method of claim 15, wherein the triboelectric sensor is affixed to an inner surface of clothing worn by the person, and the at least one other surface comprises skin of the person.

17. The method of claim 15, wherein the triboelectric sensor is interwoven into clothing worn by the person.

18. The method of claim 15, wherein the triboelectric sensor is affixed to an outer surface of clothing worn by the person.

19. The method of claim 15, wherein the triboelectric sensor is incorporated with a hip protector adorned by the person.

20. A fall detection system comprising:
a triboelectric sensor that is securable to a portion of a person's body or is affixed to clothing worn by the person, wherein the triboelectric sensor includes at least one electrode that defines a surface;
readout circuitry that detects charge at the at least one electrode caused by physical contact between the surface of the at least one electrode and at least one other surface, wherein the other surface includes one or more of the person's skin or a ground surface;
one or more output components; and
logic that receives, from the readout circuitry, a first signal indicative of the detected charge at the at least one electrode and, based at least in part on the first signal, provides, at one or more of the output components, output indicative of a detected fall.
